(19) United States
(12) Patent Application Publication
(10) Pub. No.: US 2022/0133263 A1
(43) Pub. Date: May 5, 2022

(54) MRI-COMPATIBLE IMPLANTABLE WIRELESS DIAGNOSTIC AND THERAPEUTIC ULTRASOUND

(71) Applicant: THE JOHNS HOPKINS UNIVERSITY, Baltimore, MD (US)

(72) Inventors: Chad R. GORDON, Cockeysville, MD (US); Micah BELZBERG, Baltimore, MD (US); Netanel BEN-SHALOM, Baltimore, MD (US); Amir MANBACHI, Baltimore, MD (US); Mehran ARMAND, Baltimore, MD (US)

(21) Appl. No.: 17/435,077

(22) PCT Filed: Feb. 27, 2020
(86) PCT No.: PCT/US2020/020222

§ 371 Date: Aug. 31, 2021

Related U.S. Application Data
(60) Provisional application No. 62/812,535, filed on Mar. 1, 2019.

(51) Int. Cl. A61B 8/12; A61B 8/08; A61B 8/00; A61N 7/00

(52) U.S. Cl. CPC A61B 8/12; A61B 8/0808; A61B 8/4472; A61B 8/4488; A61N 2007/0078; A61B 8/56; A61N 7/00; A61N 2007/0052; A61N 2007/0047; A61B 8/54

(57) ABSTRACT

An implantable ultrasonic transducer device for capturing radiographic and biometric data is provided. The implantable ultrasonic transducer device includes a transducer array configured to provide ultrasonic waves to a target area and to obtain reflected ultrasonic waves from the target area; a controller electrically coupled to the transducer array and configured to provide one or more control signals to the transducer array to control one or more modes of operation of the transducer array; and an antenna electrically coupled to the controller and configured to wireless transmit and receive data from an external device, wherein the transducer array, the controller, and the antenna are completely contained within a body cavity of a patient and an activation surface of the transducer array is positioned in physical contact with a portion of a treatment area of the patient with no air gap between the activation surface and the treatment area.

### RELATED APPLICATION

This application claims the benefit of, and priority to, U.S. Provisional Patent Application No. 62/812,535 titled “MRI-COMPATIBLE IMPLANTABLE WIRELESS DIAGNOSTIC AND THERAPEUTIC ULTRASOUND,” and filed on Mar. 1, 2019, which is hereby incorporated by reference in its entirety.

### FIELD

This disclosure relates generally to a magnetic resolution imaging (“MRI”)-compatible, implantable, wireless, diagnostic and/or therapeutic ultrasound device to improve neurosurgical care.

### BACKGROUND

In the field of neurosurgery, neuroplastic surgery, craniofacial plastic surgery, ENT-head and neck surgery, neurology, and reconstructive ophthalmologic surgery, imaging and biometric data play robust roles in diagnosis, monitoring and surveillance of neurological pathologies following craniotomy or intra-cranial surgery. Current imaging methods, such as CT and MRI, which are the current standard-of-care, are both expensive, time-intense, resource consuming, and not always readily available. More importantly, CT scans and MRI images are taken in isolated, static form as opposed to providing a surgeon a high-yield, cumulative view with respect to time (i.e. showing 30 consecutive daily images all constructed into one image with respect to time and change)—which limits the decision-making capacity of each healthcare provider upon looking at the image. For example, following brain tumor resection, an MRI taken three months later is often confusing since there are challenges deciding whether one see tumor re-growth, blood accumulation, or radiation therapy-induced changes. Thus, there is room for major improvement in the way we image our neurological patients status-post either craniotomy or intra-cranial surgery like cranioplasty (i.e. skull reconstruction).

Craniotomy (i.e. opening of the skull for the purpose of brain surgery), craniectomy (i.e. removal of cranial bone to allow for brain swelling, to remove for tumor, to treat infection and/or sterile resorption) or cranioplasty (i.e. to reconstruct a pre-existing skull defect with autologous bone and/or man-made implant) are commonly performed by neurosurgeons, neuroplastic surgeons, and craniofacial plastic surgeons. There are numerous indications in adults and children for craniotomy or craniectomy including congenital skull deformities, acquired skull asymmetry/deformity, head trauma, brain/skull malignancy, hydrocephalus, neuromodulation for epilepsy/movement disorders, and/or hemorrhage evacuation following stroke. Following removal, the missing skull may be repaired by means of a cranioplasty using a variety of available options. In some instances, a prefabricated customized cranial implant is used in situations where autologous bone is either unavailable or suboptimal. Regardless, all three types of surgery—craniotomy, craniectomy, and cranioplasty—have significant risk and need for intra-cranial imaging to observe and/or identify the potential for post-operative complications related to various aspects of the brain underneath. Currently, the only options are to acquire a CT scan or MRI.

In procedures involving the thoracic or the abdominal region, hand held, external, wireless diagnostic ultrasounds are typically available for general clinical use, but these devices are neither implantable nor MRI compatible. For example, the emergency room/trauma teams often use rapid ultrasound imaging of the abdomen to visualize any potential complications for which require surgical intervention.

Accordingly, what is needed is a MRI-compatible device that overcomes the above-noted deficiencies and is implantable within the intra-cranial space following craniotomy, craniectomy, and/or cranioplasty. Thus, a device to satisfy the current need would likely be 1) thinly-shaped and 2) designed for rigid-fixation to either the undersurface of own's own bone flap being re-inserted at the end of common-day craniotomy or to the undersurface of a man-made skull implant in instances when the bone flap is unavailable.

### SUMMARY

In accordance with examples of the present disclosure, an implantable ultrasonic transducer device for capturing radiographic and biometric data is provided. The implantable ultrasonic transducer device comprises a transducer array configured to provide ultrasonic waves to a target area and to obtain reflected ultrasonic waves from the target area; a controller electrically coupled to the transducer array and configured to provide one or more control signals to the transducer array to control one or more modes of operation of the transducer array; and an antenna electrically coupled to the controller and configured to wireless transmit and receive data from an external device, wherein the transducer array, the controller, and the antenna are completely contained within a body cavity of a patient and an activation surface of the transducer array is positioned in physical contact with a portion of a treatment area of the patient with no air gap between the activation surface and the treatment area.

In some examples, the body cavity is an intracranial region and the treatment area is a portion of a brain of the patient or the body cavity is a thoracic or abdominal intracranial region, and the treatment area is an organ other than the brain.

In some examples, the implantable ultrasonic transducer device further comprises a power source electrically coupled to the transducer array and the antenna and configured to provide power to the transducer array. In some examples, the implantable ultrasonic transducer device, further comprises a device housing configured to house the transducer array, the controller, the antenna, and the power source.

In some examples, the data transmitted from the antenna is real-time imaging and biometric data associated with the target area.

In some examples, a first mode of the one or more modes of operation is therapeutic and a second mode of the one or more modes of operation is diagnostic.

In some examples, the one or more control signals provided by the controller is configured to activate the transducer array to produce ultrasonic wave of sufficient amplitude, phase, duration to provide a modulation of a brain, a disruption of a blood-brain barrier, or both.

In some examples, the transducer array comprises a first set of transducers configured to provide for diagnostic of the target area and a second set of transducers configured to provide for therapy of the target area.

In some examples, the controller is configured to provide a first set of control signals to the first set of transducers and a second set of control signals to the second set of transducers, wherein the first set of control signals differ from the second set of control signals by causing the first set of transducers to operate in a diagnostic mode of operation and by causing the second set of transducers to operate in a therapeutic mode of operation.

In some examples, the device housing is molded to conform to a shape of a body being implanted including head, chest, or abdomen. In the head example, the shape or curvature is that of a skull.

In some examples, the device housing is composed of: MRI-compatible titanium or any other varying combination of man-made biomaterials safe for MRI imaging.

In some examples, the transducer array is composed of: piezoelectric crystals, ceramic or 1-3 composite.

In some examples, the implantable ultrasonic transducer device further comprises an acoustic mirror that is coupled with the transducer array and configured to acoustically reflect the ultrasonic waves back into the target area.

In some examples, an activation axis of a transducer element of the transducer array is arranged substantially perpendicular to the target area or is arranged substantially parallel to the target area.

In some examples, the transducer array is configured to steer the ultrasonic waves in one or more directions. In some examples, the transducer array is shaped to steer the ultrasonic waves in one or more directions.

In some examples, the biometric data is pressure and blood flow rate associated with the target area.

In some examples, the antenna is configured to provide the biometric and radiographic data to a 3D or 4D viewing system.

In some examples, the transducer device is bio-compatible and magnetic resonance imaging (MRI)-compatible.

In some examples, a dura covering of a brain of the patient is sewn directly to the implantable ultrasonic transducer device in order to optimize ultrasound efficacy.

In some examples, the diagnostic comprising imaging of at least a portion of the target area.

In some examples, the one or more first control signals or the one or more second control signals provided by the controller are configured to activate the transducer array to produce ultrasonic wave of sufficient amplitude, phase, duration to active acoustic dye and record resultant information comprising at least one of: pathophysiologic data, positional data, and metabolic data.

In some examples, the implantable ultrasonic transducer device is partially recessed within an intact skull, one's own bone flap following craniotomy and replacement, and/or cranial implant.

In some examples, the implantable ultrasonic transducer device contains space for placement of components or devices with synergistic applications comprising at least one of: therapeutic delivery, neuroactivity recording, and hydrocephalus cerebrospinal fluid shunting.

In some examples, the transducer array is placed at a tip of an articulating snake, wherein the articulating snake is MRI-compatible. The articulating snake can be manipulated wirelessly in real time or programed to move in a predetermined sequence. The articulating snake can contain a hollow corridor for passage of objects comprising at least one of: biopsy instruments, high-definition camera, therapeutic delivery systems, and acoustic dye injection. The articulating snake length can be adjusted to place the articulating snake within deep resection cavities.

According to examples of the present disclosure, a computer-implemented method of providing medical services to a patient is provided. The computer-implemented method comprises obtaining, by a wireless antenna of an implantable ultrasonic transducer device, one or more first control signals configured to control one or more modes of operation of a transducer array from a client device; providing, by a controller of the implantable ultrasonic transducer device, one or more second control signals based on the one or more first control signals to a transducer array, wherein the transducer array comprises at least one of diagnostic transducer elements, therapeutic transducer elements, or both; obtaining, by the controller, one or more response signals based on activation of the transducer array; and providing, by the wireless antenna, the one or more response signals to the client device for evaluation, wherein the transducer array, the controller, and the wireless antenna are enclosed within the implantable ultrasonic device and completely contained within a body cavity of a patient and an activation surface of the transducer array is positioned in physical contact with a portion of a treatment area of the patient with no air gap between the activation surface and the treatment area.

In some examples, the client device can be a smart phone, a laptop computer, a table computer, or a desktop computer. In some examples, the one or more first control signals are obtained by an application operating on the client device.

In some examples, the one or more second control signals are determined by the controller to provide an appropriate transducer activation parameter for an operation mode of the one or more modes of operations.

According to examples of the present disclosure, a computer-implemented method of providing medical services to a patient via an implantable ultrasonic transducer device is provided. The computer-implemented method comprises opening, on a client device, an application to control the implantable ultrasonic transducer device; selecting, in the application, a patient from among a plurality of patients for treatment; providing, by the application, one or more control signals configured to control one or more modes of operation of the implantable ultrasonic transducer device; and obtaining, by the application, one or more response signals based on activation of the implantable ultrasonic transducer device for evaluation, wherein the implantable ultrasonic device is completely contained within a body cavity of a patient and an activation surface of a transducer array is positioned in physical contact with a portion of a treatment area of the patient with no air gap between the activation surface and the treatment area.

Neuro-oncology: Following the resection of brain tumors, the current standard-of-care requires strict post-resection surveillance with periodic imaging to rapidly diagnose any recurrence given the limited room available for expansion within the skull—most often at a time interval of every three months, based primarily on tumor pathology and overall risk for recurrence. Across the board, MRI is the preferred imaging modality for brain (versus CT scan, which is better for bone) and is therefore performed regularly to evaluate the brain (and its surrounding structures) following resection, identifying for tumor re-growth, parenchymal edema and/or tissue necrosis—especially in instances of adjuvant brain radiation and/or chemotherapy. Of note, MRI imaging with contrast injection may enhance solid tumors and metastasis but has little diagnostic value of the actual tissue architecture and in non-enhancing lesions such as low-grade gliomas.

MRI is expensive, time-consuming, requires manpower and resources, is not readily available, cannot be administered remotely, and generates only a static image of the brain at a single time point. By contrast, ultrasound is inexpensive, non-ionizing, may be performed at bedside, and could permit real-time, remote, wireless, interactive image acquisition if this invention is successful. Ultrasound is widely used in the setting of neurosurgery for localizing low grade gliomas and other intrinsic brain tumors, assessing tumor volume, and evaluating tumor resection. However, the acoustic properties of skull bone limit transcranial ultrasound, which is why there is a promising opportunity to create a novel, implantable device for rigidly fixation to the undersurface of one's bone flap during craniotomy.

Intracranial Ultrasound (IUS), as described here for the first time, will be an implantable imaging and sampling device than can generate dynamic images and acquire biometric data over multiple time points—thereby providing more efficient and more informative post-operative surveillance. The data captured by the IUS will be wirelessly transmitted to the treating physician (or interested patient/family member) thereby simplifying and expediting the process of post-operative follow-up and brain imaging surveillance. Machine learning may be applied to this data for a deeper analysis or to automatically generate alerts in the case of suspected tumor regrowth or treatment failure. With more data and more data points, IUS will be able to discover tumor re-growth sooner, utilize acoustic dies to better evaluate pathophysiology, and facilitate biopsy of suspicious lesions by using a robotic arm extension following remote control. Subsequently, IUS will also be able to perform therapeutic interventions by means of “focused” ultrasound (different frequency and intensity, as opposed that used in “imaging” ultrasound) for such applications as enhancing intraparenchymal drug delivery (i.e. directly into brain, as opposed to a systemic/intravenous route), thermal ablation in eloquent areas of brain non-amenable to resection, and/or targeted blood brain barrier disruption for enhanced local drug delivery.

Vascular Neurosurgery: Neuro-pathologies such as stroke, cerebral aneurysms, vascular malformations, and intracranial hemorrhages require close surveillance with multiple CT scans (+/−MRI imaging as well) in the immediate post-operative period (especially when there are mental status changes, severe headaches, etc.) to assess for re-bleeding, brain edema, midline shift, and/or structural brain changes. The frequency of these CT scans are determined by a combination of factors including the patient's clinical condition, physician preference and guidelines.

In contrast, IUS implantation (to the undersurface of the bone flap) will allow for immediate, continuous, dynamic collection of pertinent data related to brain anatomy by way of a self-supplied battery source. Endpoints of relevance include diagnosis brain edema, midline shift, re-bleed, cerebral blood flow and ventricular size, and intra-cranial pressure changes. Compared to CT radiographic imaging, IUS information will be collected faster, remotely (literally, from anywhere in the world), without ionizing radiation exposure, and can be transmitted instantaneously to any healthcare provider(s)—thereby in sharp contrast to the time consuming, labor-intensive and costly process of transporting a patient emergently to a radiology suite for CT scan or MRI. Additionally, through machine learning, the IUS can be be trained to recognize findings suggestive of edema, increased intracranial pressure, re-bleeding, midline shift and hydrocephalus and automatically alert the patient and/or healthcare provider unlike any other available technology on the market today. Plus, neurological injury from pressure changes or ischemia is highly time dependent, and so intuitively-speaking, a remote diagnosis from IUS for brain changes is most valuable in terms of saving time, when compared to having to find a local hospital, rush into the emergency room, and to obtain an emergent CT scan/MRI.

Pediatric Neurosurgery: Common intracranial pathologies in the pediatric population include hydrocephalus, brain neoplasms, spinal cord tumors, craniofacial disorders like craniosynostsosis, and epilepsy. Such chronic diseases require lifelong radiographic imaging surveillance thereby subjecting children to an increased amount of exposure to ionizing radiation and frequency hospital/clinical visits—which has a negative effect of unknown consequence and therefore under current scrutiny. Implantable IUS in pediatric patients with chronic neuropathology provides, lifelong im. However, since there is no IUS available, pediatric neurosurgeons have no choice than to use ionizing radiation for pediatric brain assessment, but without question, would be most in favor of using remote IUS imaging when and if available—especially because there will be no radiation of concern thereby removing the common frustrating barriers for repeat imaging. Furthermore, aging surveillance, as the child continues to grow, will be performed remotely with IUS and able to stay in indefinitely—employing a wireless charger like a cell phone—which will be worn at nighttime and in the shape of a headphone or pillowcase. With daily charging, the IUS can be turned on when patients have acute complaints and the image can be immediately sent to the treating physician for further evaluation—thereby saving the healthcare system millions of dollars each and every year.

For example, pediatric hydrocephalus is common and requires an emergency room visit and CT scan each and every time a parent is concerned about their child's affect and/or mood. But, most concerning, is the fact that 9 out of 10 visits turn out to be wasted. Therefore, imagine the incredible impact that the IUS will have on the pediatric neurosurgery population.

Trauma: Military and civilian head trauma patients require close intracranial monitoring for early recognition of secondary brain damage following a brain insult, concussion, and/or traumatic head injury. Traumatic hematomas require immediate evacuation and ICP monitoring, or else serious sequelae such as death is possible. Sedated and intubated military patients from the battlefield, along with civilian neurotrauma patients in intensive care units, both receive daily head CTs. Depending on their Glacgow Coma Score (GCS), neurological exam, and/or brain function in terms of extremity movement, they may require external ICP monitors (i.e. “bolt”). These need to be carefully managed since they are at risk for infection thereby requiring surgical replacement. Mainly because it requires a wire and connection placed from the external computer, through the scalp, and into the brain.

Implantable IUS, placed at time of their emergent/urgent surgery, will provide a diagnostic tool for detection of brain shift, edema, re-bleed and expectant secondary damage. Furthermore, IUS will provide the caregiver continuous, intra-cranial pressure monitoring and can alert the patient or healthcare provider if a problem is suspected.

### BRIEF DESCRIPTION OF THE DRAWINGS

Various features of the embodiments can be more fully appreciated, as the same become better understood with reference to the following detailed description of the embodiments when considered in connection with the accompanying figures, in which:

FIG. 1 shows a diagrammatic view of device according to examples of the present disclosure;

FIG. 2 shows an alternative array for integrated therapeutic elements according to examples of the present disclosure;

FIGS. 3A, 3B, 3C, and 3D show diagrams of electronic and mechanical steering of transducer elements of transducer array layer, which can be arrayed in multiple configurations according to examples of the present disclosure;

FIGS. 4A, 4B, 4C, 4D, 4E, 4F, 4G, 4H, 4I, 4J, 4K, 4L, and 4M show a section through transducer showing different configurations according to examples of the present disclosure;

FIG. 5 shows device components may be reconfigured in numerous rearrangements according to examples of the present disclosure;

FIGS. 6A, 6B, 6C, and 6D shows four different device configurations according to examples of the present disclosure;

FIG. 7 shows an orthogonal view of transducer variant design according to examples of the present disclosure;

FIG. 8 shows an orthogonal view of device components arranged within custom cranial implant, skull or stand-alone device according to examples of the present disclosure;

FIG. 9 shows an oblique view of skull showing device embedded within a cranial implant or as a stand-alone device according to examples of the present disclosure;

FIG. 10 show another view of cranium with cranial implant, according to examples of the present disclosure.

FIGS. 11A, 11B, 11C and 11D show an oblique view of skull showing variant designs embedded within a cranial implant or as stand-alone devices according to examples of the present disclosure;

FIGS. 12A and 12B show sectional views of transducer variant design according to examples of the present disclosure;

FIGS. 13A and 13B show sectional views of transducer variant design according to examples of the present disclosure;

FIGS. 14A, 14B, and 14C show sectional views of transducer variant design according to examples of the present disclosure;

FIG. 15 shows a computer-implemented method of providing medical services to a patient according to examples of the present disclosure;

FIG. 16 shows a computer-implemented method of providing medical services to a patient via an implantable ultrasonic transducer device according to examples of the present disclosure; and

FIG. 17 is an example of a hardware configuration for a computer device according to examples of the present disclosure.

### DESCRIPTION OF THE EMBODIMENTS

Reference will now be made in detail to example implementations, illustrated in the accompanying drawings. Wherever possible, the same reference numbers will be used throughout the drawings to refer to the same or like parts. In the following description, reference is made to the accompanying drawings that form a part thereof, and in which is shown by way of illustration specific exemplary embodiments in which the invention may be practiced. These embodiments are described in sufficient detail to enable those skilled in the art to practice the invention and it is to be understood that other embodiments may be utilized and that changes may be made without departing from the scope of the invention. The following description is, therefore, merely exemplary.

Generally speaking, examples of the present disclosure provides for an MRI-compatible, remote ultrasound transducer with a self-contained, power source imbedded underneath one's own bone flap (or recessed underneath a cranial implant if and when the patient's own bone is not available). The system, by sitting directly in contact with the brain/dura, can capture and wirelessly transmit radiographic images and biometric data to an external device or software application—which is in great contrast to expensive, time consuming, and not readily-available MRI and ionizing CT radiographs. The IUS system will provide a novel means of acquiring real-time, non-ionizing, continuous, remote, post-operative monitoring and long-term surveillance of the brain and all related structures.

Aspects of the present disclosure optimizes the relationship between a thinly-shaped, curved, wireless, remote, ultrasound imaging device with Bluetooth connectivity and the growing world of functional neurological implants in synergy—thereby making it possible to integrate the disclosed IUS component within a position on the undersurface of own's own bone flap device (or cranial implant if required). Such improvements exploit the benefits of direct access to the brain and ideal anatomical location/proximity provided by the disclosed devices placed in direct contact with the brain.

In accordance with examples of the disclosure, the IUS implant can be fabricated from a wide array of MRI-compatible or MRI-safe biomaterials including medical-grade titanium. This material allows for both rigid fixation (zero motion once screwed into place on the undersurface of cranial bone) and novel inspection of the brain at anytime and anywhere. As will be explained below in greater detail, it also allows for the critical transmission of vital imaging with minimal distortion, such as ultrasound waves for brain pathology detection, ventricle size change, and blood flow interruption.

Based upon the MRI-compatible implantable device used in conjunction with examples of the present disclosure, the disclosed functional neurosurgical implant may be useful in the monitoring and/or treatment of various patient conditions such as epilepsy, movement disorders, chronic pain, spasticity, cerebral palsy, multiple sclerosis, spinal cord injury, traumatic brain injury, attention-deficit/hyperactivity disorder, autism, etc.—and the potential to obtain supra-normal levels of brain monitoring in both military and civilian situations following head trauma. Furthermore, incorporation of IUS imaging devices to one's own bone flap following brain tumor craniotomy could help to provide ongoing tumor bed monitoring for early detection of disease recurrence, and may impact the chances of life versus death.

In examples, the MRI-compatible IUS device, which emits/receives ultrasonic waves by altering the frequency and power of an individual transducer, the device may be used for diagnostic and or therapeutic purposes as a standalone implantable disc attached to the undersurface of the native bone flap. The diagnostic ultrasound obtains radiographic images and biometric data such as pressure and flow rate. The therapeutic ultrasound is designed to emit ultrasonic energy to specific targets for applications such as brain modulation and disruption of the blood brain barrier (to enhance medicine delivery). The diagnostic and therapeutic transducers can work together to target and re-target the array elements, in sync with an included drug delivery chamber, to enhance local drug “convection” (of note, convection-enhanced delivery of medicine has been shown to have different outcomes versus just dripping medicine into the brain due to differences in drug perfusion) into the surrounding tumor cavity by breaking blood brain barrier [as described by previous invention by Gordon, Weingart, et al filed in 2018 entitled “Magnetic Resonance Imaging Compatible, Convection-Enhanced Delivery Cranial Implant Devices and Related Methods U.S. Provisional Patent Application No. 62/692,111, which is hereby incorporated by reference in its entirety. The diagnostic and or therapeutic ultrasound transducer may be mechanically or electronically steered using a remote control mobile app. The Bluetooth connection enables the device to be re-programed wirelessly and will allow data to be sent from the implanted IUS device to an external device or software application. Data obtained and device settings may be read by, adjusted by and sent to the patient or a healthcare provider. The components of the device may be re-arranged to permit assembly within 1) the undersurface of a patient's own bone, 2) a cranial implant made of various sizes, shapes and biomaterials (when and if bone flap not present) or 3) to permit implantation elsewhere in the body. Also included is an implantable and or external power source which may be rechargeable, in the shape of a wireless headphone or pillow case.

In one non-limiting example, the system can be embodied in a miniaturized ultrasound inserted into the undersurface of a custom-fabricated cranial implant which is surgically affixed to the surrounding skull or attach to patients own bone by screws and plates. The implant includes, among other things and as discussed further below, an ultrasonic transducer (extruding from the undersurface to allow for direct contact with brain and/or dura), a microcontroller, and antenna. The implant can wirelessly communicate with a client device. The client device can include a software application that can control, at least in part, the ultrasonic transducer to produce immediate non-ionizing ultrasound images and biometric data which can be wirelessly transmitted to the client device and additional one or more other devices. Thus, the implant provides continuous details about the intracranial space whenever needed.

The system can also provide for real-time, bedside monitoring and chronic surveillance. The system can be used immediately after the surgery to look intra-cranially when any symptoms or concerns arise. The IUS can be connected to any hospital system either in intensive care unit, regular hospital floor bed, or at home using one's own mobile device. Essentially, the data transmitted from the IUS will be device-agnostic. This enhanced visualization and biometric data technique not only results in more effective detection of complication but also guides long-term surveillance for various brain pathologies, thereby eliminating the risks and cost/radiation burden associated with computer tomography (CT) scanning.

By way of an included reservoir, the system can be used with injectable acoustic dyes to visualize, detect or quantify aspects of pathophysiology or anatomy. Such dyes may be injected systemically or locally. Applications include lesion marking, anatomical enhancement and quantification of pathophysiology. A remote controlled snake robot will be added to the IUS in instances where deep intraparenchymal extension may be needed.

Examples of the present disclosure provides for an MRI-compatible and implantable device that allows for a wireless connection to an external device or devices. The present devices leaves limited externally visible signs of implantation since its on the undersurface of the patient's own skull bone flap, or cranial implant. The present device is operable to collect dynamic real-time diagnostic radiographic images and biometric data. The images and data can be manipulated using the native software application. The present device provides depth-related information. The present device provides for components which may be reconfigured for placement in other locations within and on the body.

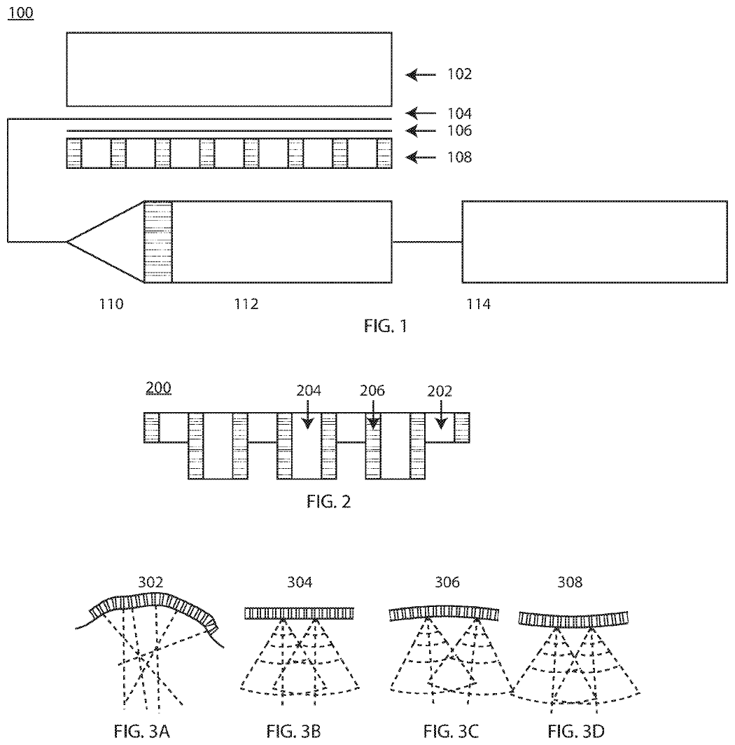

FIG. 1 shows a diagrammatic view of device 100 according to examples of the present disclosure. Device 100 includes backing layer 102 for directing sound. For example, backing layer 102 can be made of rubber. Device 100 also includes electrodes 104, metal layer 106 made of gold or copper, and transducer array layer 108. Transducer array layer 108 is electrically coupled to multiplexer 110, microcontroller 112, and battery 114. Transducer array layer 108 can include a piezoelectric layer, which can be composed of quartz, ceramic, or a 1-3 composite material, that can provide for center frequencies in the range of 200 kHz to 1 MHz. Piezo-ceramic materials that can be used in the piezoelectric layer can be characterized by having good electrical to mechanical conversions capabilities, but relatively low internal damping.

FIG. 2 shows an alternative transducer array layer 200 for integrated therapeutic elements according to examples of the present disclosure. Transducer array layer 200 comprises a repeating pattern of diagnostic transducer elements 202 and therapeutic transducer elements 204 separated by insulator elements 206. In some examples, therapeutic transducer elements 204 can have a larger dimension than diagnostic transducer elements 202 that can be concentrated more centrally to provide increased image resolution. Diagnostic transducer elements 202 can be controlled to produce ultrasonic energies sufficient to provide one or more ultrasound images. Therapeutic transducer elements 204 can be controller to produce ultrasonic energies sufficient to provide a therapeutic procedure, such as, but are not limited to, a disruption of the blood-brain barrier, lesion ablation, neuromodulation/epilepsy control, dissolution of thrombi, targeted therapeutic activation, movement disorder prevention, and pain management. Such bioeffects may be achieved by varying beam frequency, pulse length, power and intensity.

FIGS. 3A, 3B, 3C, and 3D show diagrams of electronic and mechanical steering of transducer elements of transducer array layer 108, 200, which can be arrayed in multiple configurations. FIG. 3A shows transducer array layer 302 in a concave-like configuration with seven transducer elements active. FIG. 3B shows transducer array layer 304 in a linear configuration with two transducer elements active. FIG. 3C shows transducer array layer 306 in a concave configuration with two transducer elements active. FIG. 3D shows transducer array layer 308 in a convex configuration with two transducer elements active. By one non-limiting example, individual transducer elements of transducer array layer 302, 304, 306, 308 can be arranged in a phased-array to modulate a focal position of the acoustic waves to provide steering of the ultrasonic beam. For example, microcontroller 112 can provide control signals to each individual transducer element to control a firing time that the individual transducer element is activated. In another non-limiting embodiment, individual transducer elements of transducer array layer 302, 304, 306, 308 can be coupled with a micro-electro-mechanical system (MEMS) and in communication with microcontroller 112 to provide control signals to each individual transducer element to control a firing time that the individual transducer element is activated.

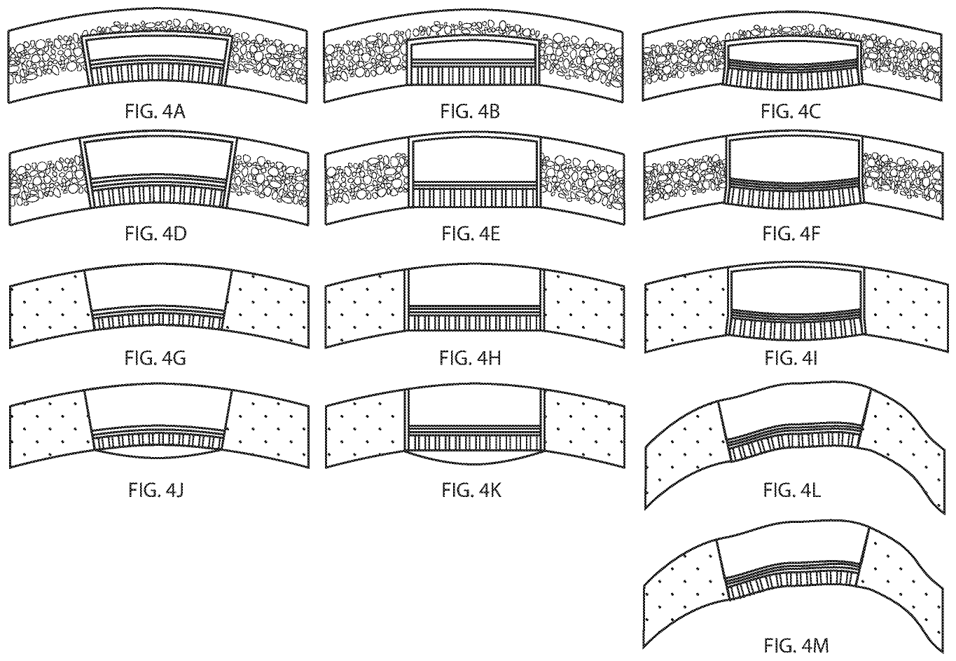

FIGS. 4A, 4B, 4C, 4D, 4E, 4F, 4G, 4H, 4I, 4J, 4K, 4L and 4M show a section through transducer showing different configurations. Transducer elements may have multiple planer relationships including concave, convex, or flat arrangement. Device may be partially recessed within skull bone flap (from the underside, not top-side—so as to not change the contour of one's outer skull) as shown in 4A, 4B and 4C. Device may be fully recessed within skull bone as shown in 4D, 4E and 4F. An acoustic lens may be placed beneath the device as shown in 4J and 4K. The elements may be configured in multiple manors. Emitters may be placed within a flexible sheet which conforms to curvature of skull as shown in 4L and 4M.

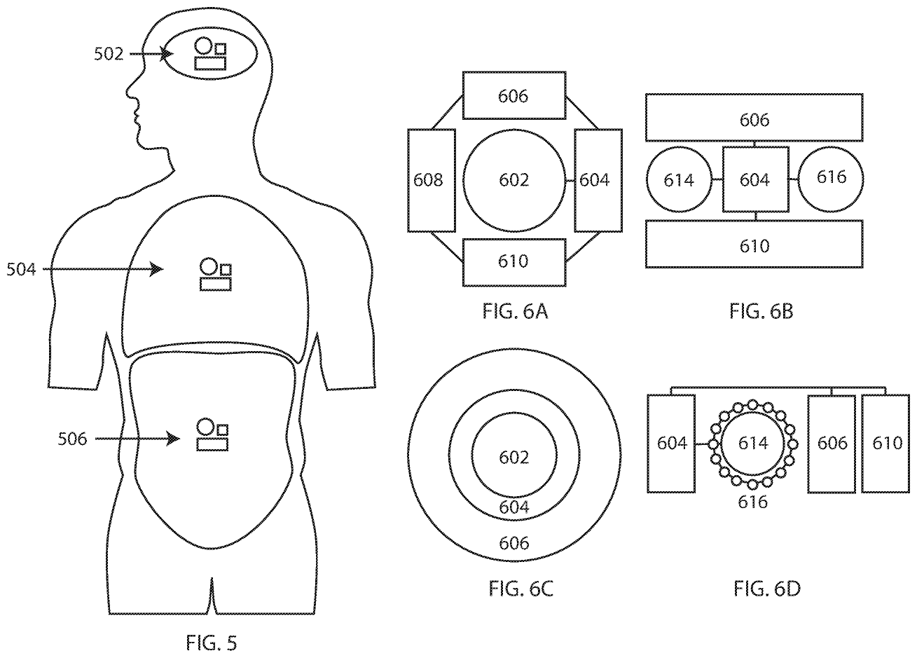

FIG. 5 shows three device placements within a patient. Device can be implanted within or on the body including head 502, thoracic cavity 504 (i.e., to possibly monitor heart function remotely), and abdominal cavity 506 (i.e. to possibly monitor fetal movement remotely).

FIGS. 6A, 6B, 6C, and 6D show four different device configurations according to examples of the present disclosure. Components of the device may be reconfigured in numerous rearrangements and can include various numbers of transducers, power sources and controller. In FIG. 6A, first transducer 602 is centrally positioned and in electrical contact with multiplexer and microcontroller 604. First transducer 602 can include both diagnostic and therapeutic transducer elements, such as in FIG. 2, or can include either diagnostic or therapeutic transducer elements. Multiplexer and microcontroller 604 is in electrical contact with first battery 606, second battery 610, and third battery 612. In FIG. 6B, multiplexer and microcontroller 604 is electrically connected to second transducer 614 and third transducer 616 on either side. Second transducer 614 can include diagnostic transducer elements and third transducer 616 can include therapeutic transducer elements, or vice versa. First battery 606 and second battery 610 are electrically connected to multiplexer and microcontroller 604 on either side. In FIG. 6C, first transducer 602 is surrounded by and electrically coupled with multiplexer and microcontroller 604, which is surrounded by and electrically coupled with first battery 606. In FIG. 6D, multiplexer and microcontroller 604 is electrically connected to first battery 606 and second battery 610. Multiplexer and microcontroller 604 is electrically connected to second transducer 614, which is electrically connected a plurality of third transducers 616. In one example, second transducer 614 includes therapeutic transducers and third transducer 616 includes diagnostic transducer, or vice versa.

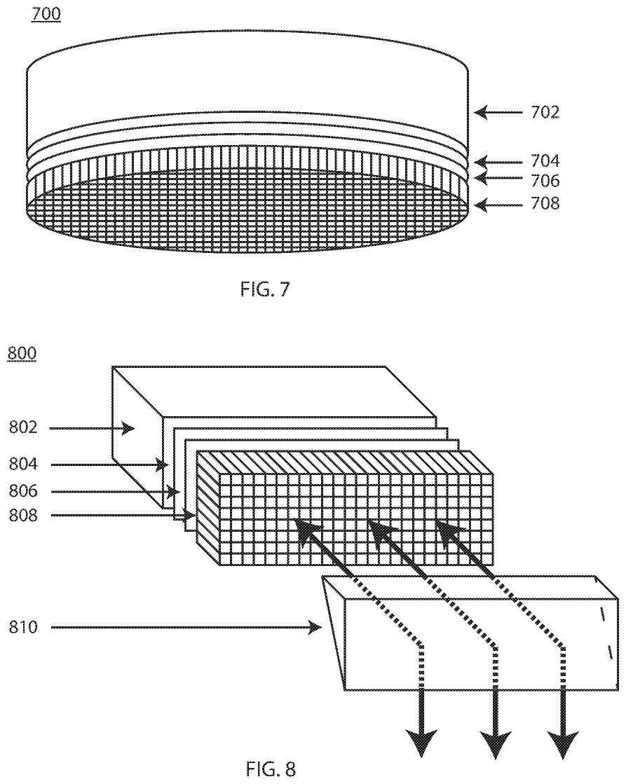

FIG. 7 shows an orthogonal view of transducer 700 showing the basic components, according to examples of the present disclosure. Transducer 700 includes backing layer 702 for directing sound. For example, backing layer 702 can be made of rubber. Transducer 700 also includes electrodes 704, metal layer 706 made of gold or copper, and transducer array layer 708. Transducer array layer 708 can include a piezoelectric layer, which can be composed of quartz, ceramic, or a 1-3 composite material, that can provide for center frequencies in the range of 200 kHz to 1 MHz. Piezo-ceramic materials that can be used in the piezoelectric layer can be characterized by having good electrical to mechanical conversions capabilities, but relatively low internal damping.

FIG. 8 shows an orthogonal view of an alternative transducer 800, according to examples of the present disclosure. Transducers 800 is arranged in an orthogonal manner than transducer 700. Transducer 800 includes backing layer 802 for directing sound. For example, backing layer 802 can be made of rubber. Transducer 800 also includes electrodes 804, metal layer 806 made of gold or copper, and transducer array layer 808. Transducer array layer 808 can include a piezoelectric layer, which can be composed of quartz, ceramic, or a 1-3 composite material, that can provide for center frequencies in the range of 200 kHz to 1 MHz. Piezo-ceramic materials that can be used in the piezoelectric layer can be characterized by having good electrical to mechanical conversions capabilities, but relatively low internal damping. Transducer array layer 808 can be arranged as square. Transducers 800 may be arranged parallel to the surface of the skull and orientated to reflect off an acoustic mirror 810.

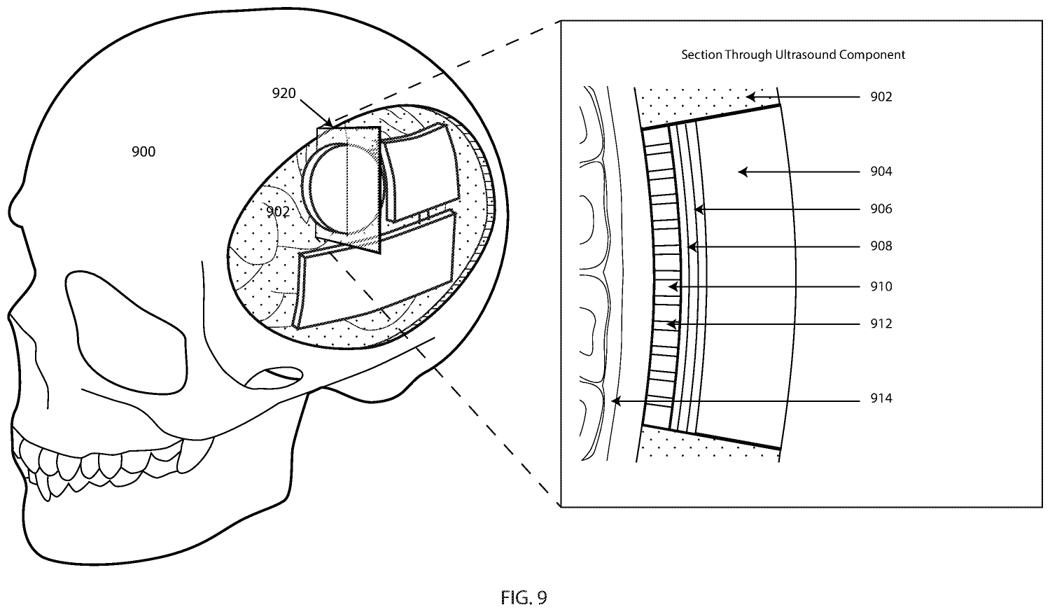

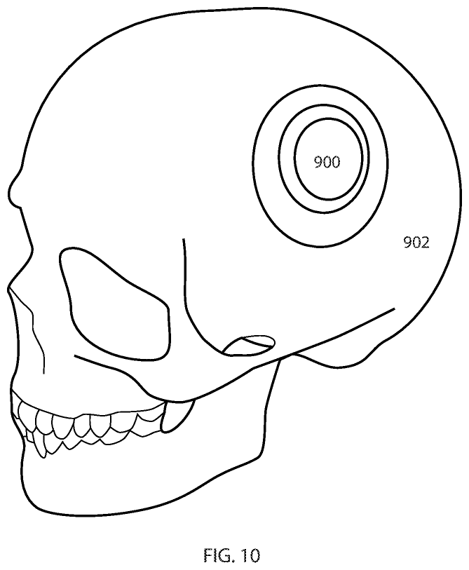

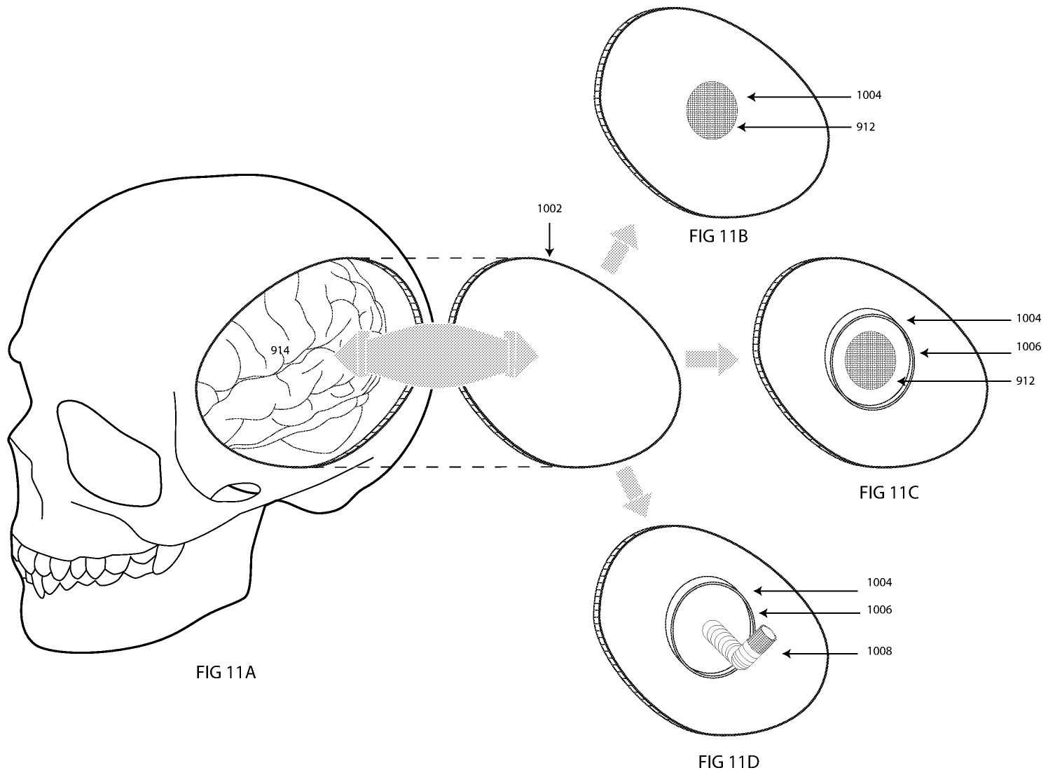

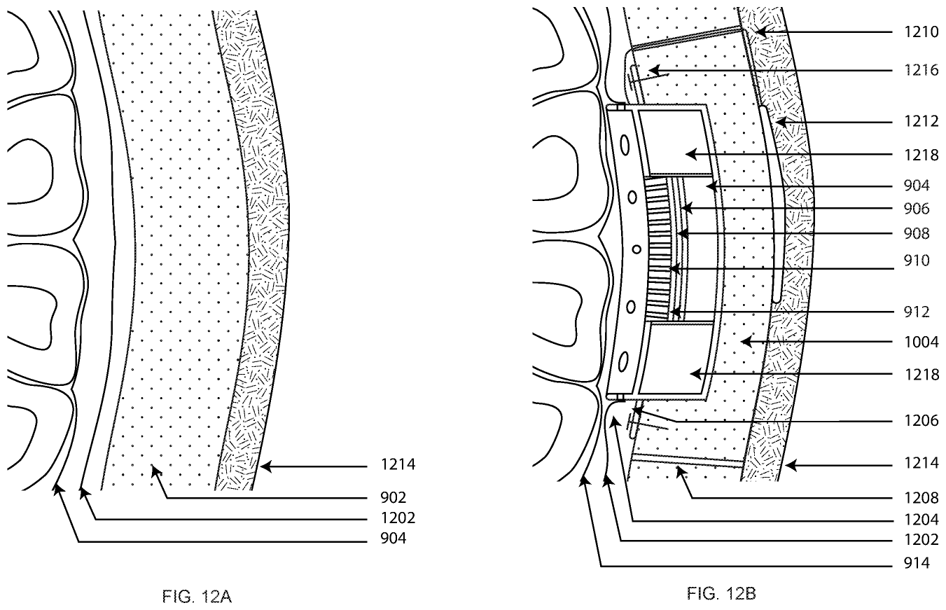

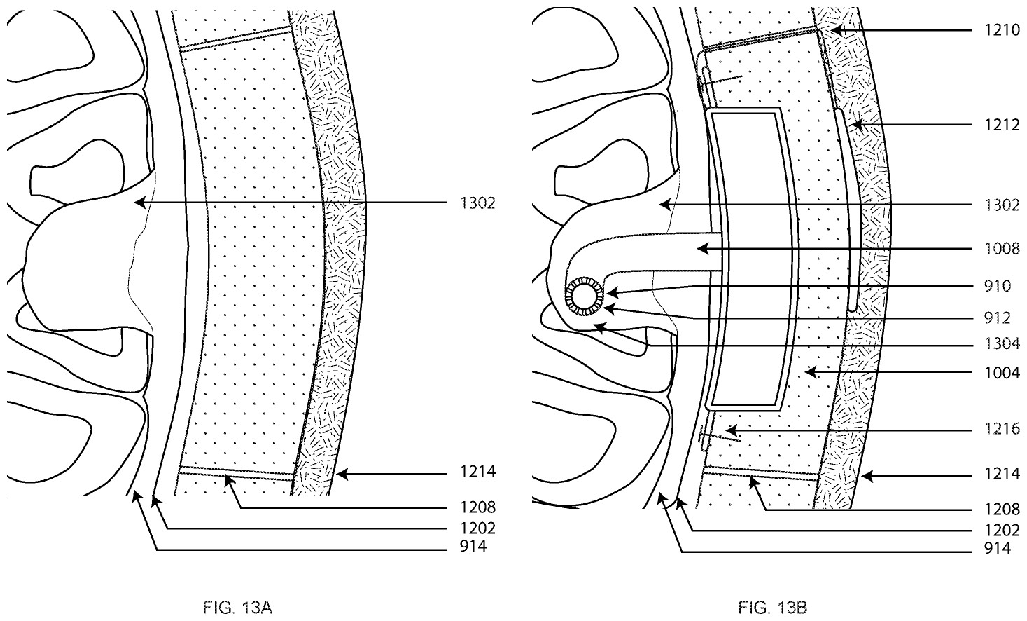

FIG. 9 shows an orthogonal view along plane 920 of device components arranged within the undersurface of a cranial implant 902 of cranium 900, according to examples of the present disclosure. FIG. 10 show another view of cranium 900 with cranial implant 902 in the absence of a synthetic implant, according to examples of the present disclosure. Cranial implant or intact skull or bone flap 902. Backing layer 904 is arranged on the inside surface of implant lid for directing sound back into the brain. For example, backing layer 904 can be made of rubber. Electrodes 906, metal layer 908 made of gold or copper, and transducer array layer 910 are arranged below backing layer 904. As discussed above with regard to FIGS. 1 and 2, transducer array layer 910 can include a piezoelectric layer, which can be composed of quartz, ceramic, or a 1-3 composite material, that can provide for center frequencies in the range of 200 kHz to 1 MHz. Piezo-ceramic materials that can be used in the piezoelectric layer can be characterized by having good electrical to mechanical conversions capabilities, but relatively low internal damping. Transducer array layer 910 includes alternating transducer elements spaced apart by insulator 912. Cranial implant 902 is arranged such that transducer array layer 910 is positioned in physical contact with brain 914.

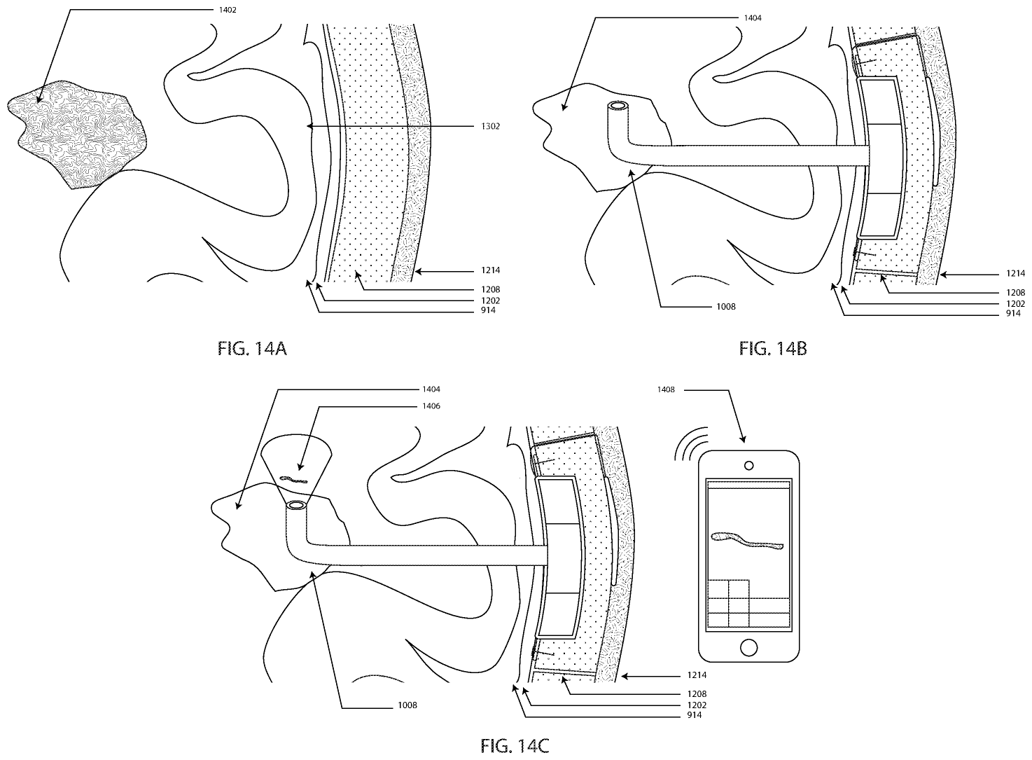

FIGS. 11A-11D show an orthogonal views of device components arranged within a craniectomy bone flap or skull implant 1002, according to examples of the present disclosure. Craniotomy bone flap or skull implant 1002 is reflected and the inner surface is shown. FIGS. 11A and 11B show another perspective of the system shown in FIG. 9 with the transducer array 912 fully recessed into the bone flap or skull implant 1004. FIG. 11C shows a transducer array 912 partially recessed within the bone flap or skull implant 1004. A cylindrical ring with perforations along the surface 1006 extends from the transducer 906 to create points for dural attachment. These perforations allow for the dura to be sewn up against the surrounding cylindrical edge thereby creating an unimpeded path between the transducer array 912 and brain 914 (i.e. ideal to have the transducers touching brain with no interference of dura). FIG. 11D shows a partially recessed device 1004 with a dural attachment ring 1006 with a robotic snake 1008 extending into the intracranial space.

FIGS. 12A and 12B show section views of a device variant design arranged within a craniotomy bone flap or skull implant 902, according to examples of the present disclosure. FIG. 12A shows the craniotomy bone flap or skull implant 902 prior to device insertion. The brain 904, dura 1202 and scalp 1214 appear intact. FIG. 12B shows a section of the device variant design partially-recessed within a bone flap or skull implant 902. To remove acoustic attenuation caused by the dura 1202, a durotomy can be performed and the edges sewn 1204 to the device's “dural attachment ring” 1006 using holes in the ring 1206. The gap between the bone flap or skull implant and skull 1208 is used to pass a chord 1210 which connects from the device to a pad for remote charging and data transfer 1212 beneath the scalp 1214. Device is attached to the skull by MRI-compatible titanium plates and 4 mm screws 1216 (which are commonly used in neurosurgery every day and are FDA-approved as being MRI-compatible). Space within the device 1218 contains power and control mechanism. Space 1218 may be enlarged to include additional components or devices such as a shunt vale, neural activity monitor or delivery mechanism for acoustic dye or a therapeutic. These components or devices can then be used synergistically.

FIGS. 13A and 13B show section views of a variant device design arranged within a craniotomy bone flap or skull implant 902, according to examples of the present disclosure. This variant design may be used in cases where a resection cavity 1302 has been created as in FIG. 13A. FIG. 13B shows a variant design in which an MRI compatible, remotely or automatically controlled, mechanically articulating snake 1008 extends from the partially recessed device. An ultrasound array 910 and 912 is placed at the tip of the snake as previously described. The snake contains a hollow corridor 1304 allowing for passage of objects such as surgical instruments, biopsy devices, therapeutic delivery systems or injection mechanism. The snake may be articulated to direct the ultrasound array or delivery corridor to various positions around the cavity. The hollow corridor may then be utilized for such applications as obtaining a biopsy, delivering a therapeutic or administering an acoustic dye. If biopsy is performed, the cytology for pathology evaluation may be aspirated transcutaneously from a reservoir imbedded nearby in direct continuity to the IUS device.

FIGS. 14A-14C show section views of a device variant design arranged within a craniotomy bone flap or skull implant 902, according to examples of the present disclosure. FIG. 14A shows a deep intraparenchymal lesion 1402. FIG. 14B shows a variant device design consisting of a robotic snake 1008 extended into the residual cavity 1404 created following deep intraparenchymal lesion excision 1402. FIG. 14C shows the robotic snake 1008 with ultrasound tip 910 & 912 detecting a recurrent lesion 1406 and wirelessly transmitting an image of the lesion to an external device 1408.

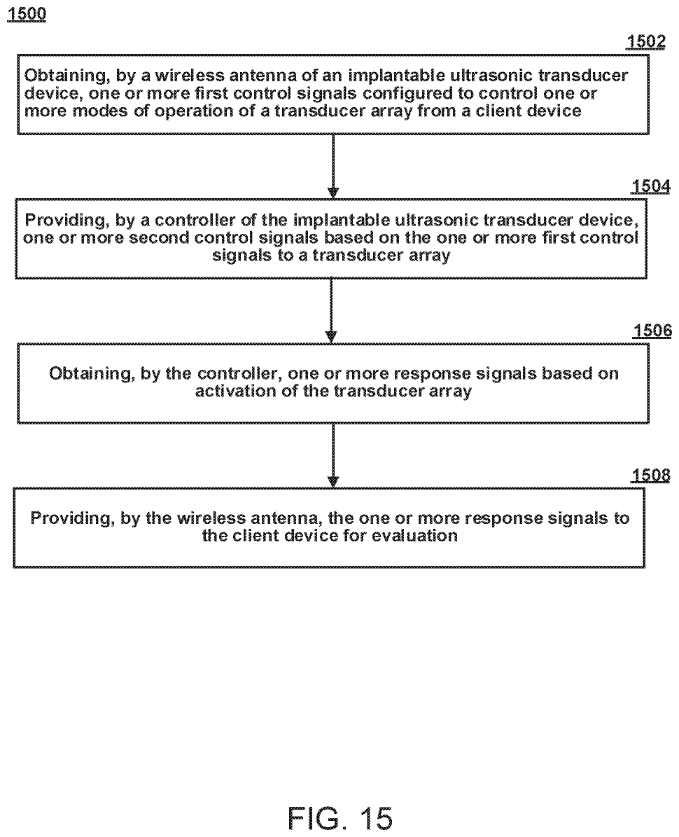

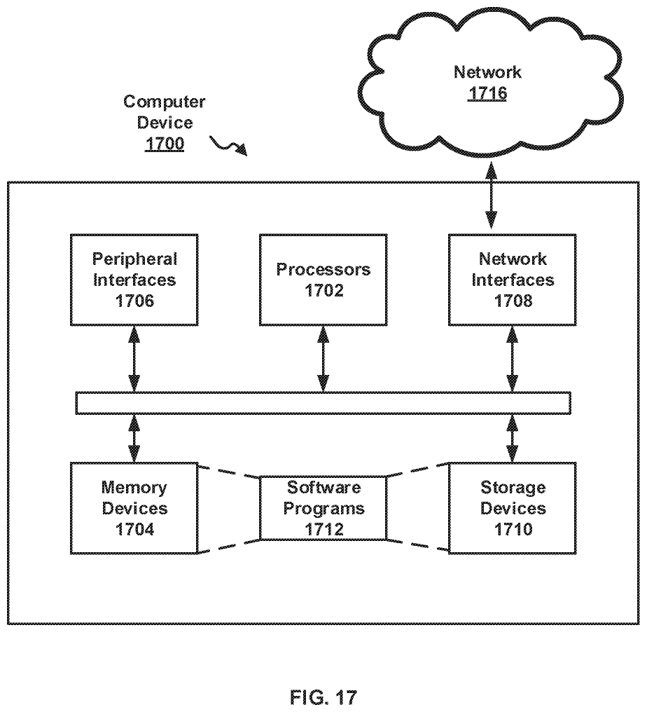

FIG. 15 shows a computer-implemented method 1500 of providing medical services to a patient according to examples of the present disclosure. The computer-implemented method 1500 begins by obtaining by a wireless antenna of an implantable ultrasonic transducer device, at 1502, one or more first control signals configured to control one or more modes of operation of a transducer array from a client device. For example, the client device can be the external device 1408, which can be a smart phone, a laptop computer, a table computer, or a desktop computer, as shown in FIG. 17 and described further below. The one or more first control signals can be obtained by an application operating on the client device, for example, the software programs 1712 on the computer device 1700.

The computer-implemented method 1500 continues by providing by a controller, e.g., microcontroller 112, of the implantable ultrasonic transducer device, at 1504, one or more second control signals based on the one or more first control signals to a transducer array. The transducer array comprises at least one of diagnostic transducer elements, therapeutic transducer elements, or both. The one or more second control signals can be determined by the controller, e.g., microcontroller 112, to provide an appropriate transducer activation parameter for an operation mode of the one or more modes of operations.

The computer-implemented method 1500 continues by obtaining by the controller, at 1506, one or more response signals based on activation of the transducer array. The computer-implemented method 1500 continues by providing by the wireless antenna, at 1508, the one or more response signals to the client device for evaluation. The transducer array, the controller, and the wireless antenna are enclosed within the implantable ultrasonic device and completely contained within a body cavity of a patient and an activation surface of the transducer array is positioned in physical contact with a portion of a treatment area of the patient with no air gap between the activation surface and the treatment area.

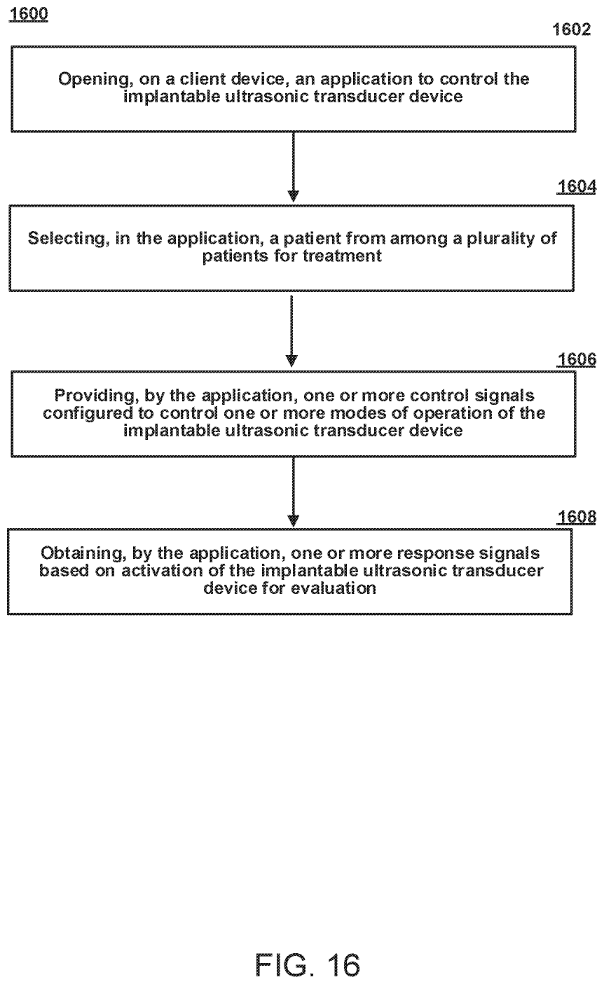

FIG. 16 shows a computer-implemented method 1600 of providing medical services to a patient via an implantable ultrasonic transducer device according to examples of the present disclosure. The computer-implemented method 1600 begins by opening on a client device, at 1602, an application to control the implantable ultrasonic transducer device. The computer-implemented method 1600 continues by selecting in the application, at 1604, a patient from among a plurality of patients for treatment. The computer-implemented method 1600 continues by providing by the application, at 1606, one or more control signals configured to control one or more modes of operation of the implantable ultrasonic transducer device. The computer-implemented method 1600 continues by obtaining by the application, at 1608, one or more response signals based on activation of the implantable ultrasonic transducer device for evaluation. The implantable ultrasonic device is completely contained within a body cavity of a patient and an activation surface of a transducer array is positioned in physical contact with a portion of a treatment area of the patient with no air gap between the activation surface and the treatment area.

FIG. 17 is an example of a hardware configuration for a computer device 1700, which can be used to perform one or more of the processes described above. The computer device 1700 can be any type of computer devices, such as desktops, laptops, servers, etc., or mobile devices, such as smart telephones, tablet computers, cellular telephones, personal digital assistants, etc. As illustrated in FIG. 17, the computer device 1700 can include one or more processors 1702 of varying core configurations and clock frequencies. The computer device 1700 can also include one or more memory devices 1704 that serve as a main memory during the operation of the computer device 1700. For example, during operation, a copy of the software that supports the above-described operations can be stored in the one or more memory devices 1704. The computer device 1700 can also include one or more peripheral interfaces 1706, such as keyboards, mice, touchpads, computer screens, touchscreens, etc., for enabling human interaction with and manipulation of the computer device 1700.

The computer device 1700 can also include one or more network interfaces 1708 for communicating via one or more networks, such as Ethernet adapters, wireless transceivers, or serial network components, for communicating over wired or wireless media using protocols. The computer device 1700 can also include one or more storage device 1710 of varying physical dimensions and storage capacities, such as flash drives, hard drives, random access memory, etc., for storing data, such as images, files, and program instructions for execution by the one or more processors 1702.

Additionally, the computer device 1700 can include one or more software programs 1712 that enable the functionality described above. The one or more software programs 1712 can include instructions that cause the one or more processors 1702 to perform the processes, functions, and operations described herein, for example, with respect to the processes of FIGS. 15 and 16. Copies of the one or more software programs 1712 can be stored in the one or more memory devices 1704 and/or on in the one or more storage devices 1710. Likewise, the data utilized by one or more software programs 1712 can be stored in the one or more memory devices 1704 and/or on in the one or more storage devices 1710.

In implementations, the computer device 1700 can communicate with other devices via a network 1716. The other devices can be any types of devices as described above. The network 1716 can be any type of network, such as a local area network, a wide-area network, a virtual private network, the Internet, an intranet, an extranet, a public switched telephone network, an infrared network, a wireless network, and any combination thereof. The network 1716 can support communications using any of a variety of commercially-available protocols, such as TCP/IP, UDP, OSI, FTP, UPnP, NFS, CIFS, AppleTalk, and the like. The network 1716 can be, for example, a local area network, a wide-area network, a virtual private network, the Internet, an intranet, an extranet, a public switched telephone network, an infrared network, a wireless network, and any combination thereof.

The computer device 1700 can include a variety of data stores and other memory and storage media as discussed above. These can reside in a variety of locations, such as on a storage medium local to (and/or resident in) one or more of the computers or remote from any or all of the computers across the network. In some implementations, information can reside in a storage-area network (“SAN”) familiar to those skilled in the art. Similarly, any necessary files for performing the functions attributed to the computers, servers, or other network devices may be stored locally and/or remotely, as appropriate.

In implementations, the components of the computer device 1700 as described above need not be enclosed within a single enclosure or even located in close proximity to one another. Those skilled in the art will appreciate that the above-described componentry are examples only, as the computer device 1700 can include any type of hardware componentry, including any necessary accompanying firmware or software, for performing the disclosed implementations. The computer device 1700 can also be implemented in part or in whole by electronic circuit components or processors, such as application-specific integrated circuits (ASICs) or field-programmable gate arrays (FPGAs).

If implemented in software, the functions can be stored on or transmitted over a computer-readable medium as one or more instructions or code. Computer-readable media includes both tangible, non-transitory computer storage media and communication media including any medium that facilitates transfer of a computer program from one place to another. A storage media can be any available tangible, non-transitory media that can be accessed by a computer. By way of example, and not limitation, such tangible, non-transitory computer-readable media can comprise RAM, ROM, flash memory, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes CD, laser disc, optical disc, DVD, floppy disk and Blu-ray disc where disks usually reproduce data magnetically, while discs reproduce data optically with lasers. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Combinations of the above should also be included within the scope of computer-readable media.

The foregoing description is illustrative, and variations in configuration and implementation can occur to persons skilled in the art. For instance, the various illustrative logics, logical blocks, modules, and circuits described in connection with the embodiments disclosed herein can be implemented or performed with a general purpose processor, a digital signal processor (DSP), an application specific integrated circuit (ASIC), a field programmable gate array (FPGA), cryptographic co-processor, or other programmable logic device, discrete gate or transistor logic, discrete hardware components, or any combination thereof designed to perform the functions described herein. A general-purpose processor can be a microprocessor, but, in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

While the teachings have been described with reference to examples of the implementations thereof, those skilled in the art will be able to make various modifications to the described implementations without departing from the true spirit and scope. The terms and descriptions used herein are set forth by way of illustration only and are not meant as limitations. In particular, although the processes have been described by examples, the stages of the processes can be performed in a different order than illustrated or simultaneously. Furthermore, to the extent that the terms “including”, “includes”, “having”, “has”, “with”, or variants thereof are used in the detailed description, such terms are intended to be inclusive in a manner similar to the term “comprising.” As used herein, the terms “one or more of” and “at least one of” with respect to a listing of items such as, for example, A and B, means A alone, B alone, or A and B. Further, unless specified otherwise, the term “set” should be interpreted as “one or more.” Also, the term “couple” or “couples” is intended to mean either an indirect or direct connection. Thus, if a first device couples to a second device, that connection can be through a direct connection, or through an indirect connection via other devices, components, and connections.

Those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

The foregoing description of the disclosure, along with its associated embodiments, has been presented for purposes of illustration only. It is not exhaustive and does not limit the disclosure to the precise form disclosed. Those skilled in the art will appreciate from the foregoing description that modifications and variations are possible in light of the above teachings or may be acquired from practicing the disclosure. For example, the steps described need not be performed in the same sequence discussed or with the same degree of separation. Likewise various steps may be omitted, repeated, or combined, as necessary, to achieve the same or similar objectives. Similarly, the systems described need not necessarily include all parts described in the embodiments, and may also include other parts not describe in the embodiments.

Notwithstanding that the numerical ranges and parameters setting forth the broad scope of the present teachings are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume negative values, e.g. −1, −2, −3, −10, −20, −30, etc.

While the preferred embodiments have been shown and described, it will be understood that there is no intent to limit the invention by such disclosure, but rather, is intended to cover all modifications and alternate constructions falling within the spirit and scope of the invention.

What is claimed is:

1. An implantable ultrasonic transducer device for capturing radiographic and biometric data, the implantable ultrasonic transducer device comprising:
a transducer array configured to provide ultrasonic waves to a target area and to obtain reflected ultrasonic waves from the target area;
a controller electrically coupled to the transducer array and configured to provide one or more control signals to the transducer array to control one or more modes of operation of the transducer array; and
an antenna electrically coupled to the controller and configured to wireless transmit and receive data from an external device,
wherein the transducer array, the controller, and the antenna are completely contained within a body cavity of a patient and an activation surface of the transducer array is positioned in physical contact with a portion of a treatment area of the patient with no air gap between the activation surface and the treatment area.

2. The implantable ultrasonic transducer device of claim 1, wherein the body cavity is an intracranial region and the treatment area is a portion of a brain of the patient.

3. The implantable ultrasonic transducer device of claim 1, wherein the body cavity is a thoracic or abdominal intracranial region, and the treatment area is an organ other than the brain.

4. The implantable ultrasonic transducer device of claim 1, further comprising a power source electrically coupled to the transducer array and the antenna and configured to provide power to the transducer array.

5. The implantable ultrasonic transducer device of claim 4, further comprising a device housing configured to house the transducer array, the controller, the antenna, and the power source.

6. The implantable ultrasonic transducer device of claim 1, wherein the data transmitted from the antenna is real-time imaging and biometric data associated with the target area.

7. The implantable ultrasonic transducer device of claim 1, wherein a first mode of the one or more modes of operation is therapeutic.

8. The implantable ultrasonic transducer device of claim 7, wherein a second mode of the one or more modes of operation is diagnostic.

9. The implantable ultrasonic transducer device of claim 1, wherein the one or more control signals provided by the controller is configured to activate the transducer array to produce ultrasonic wave of sufficient amplitude, phase, duration to provide a modulation of a brain, a disruption of a blood-brain barrier, or both.

10. The implantable ultrasonic transducer device of claim 1, wherein the transducer array comprises a first set of transducers configured to provide for diagnostic of the target area and a second set of transducers configured to provide for therapy of the target area.

11. The implantable ultrasonic transducer device of claim 10, wherein the controller is configured to provide a first set of control signals to the first set of transducers and a second set of control signals to the second set of transducers, wherein the first set of control signals differ from the second set of control signals by causing the first set of transducers to operate in a diagnostic mode of operation and by causing the second set of transducers to operate in a therapeutic mode of operation.

12. The implantable ultrasonic transducer device of claim 4, wherein the device housing is molded to conform to a shape of a body being implanted including head, chest, or abdomen.

13. The implantable ultrasonic transducer device of claim 12, wherein the shape or curvature is that of a skull.

14. The implantable ultrasonic transducer device of claim 4, wherein the device housing is composed of: MRI-compatible titanium or any other varying combination of man-made biomaterials safe for MRI imaging.

15. The implantable ultrasonic transducer device of claim 1, wherein the transducer array is composed of: piezoelectric crystals, ceramic or 1-3 composite.

16. The implantable ultrasonic transducer device of claim 1, further comprises an acoustic mirror that is coupled with the transducer array and configured to acoustically reflect the ultrasonic waves back into the target area.

17. The implantable ultrasonic transducer device of claim 1, wherein an activation axis of a transducer element of the transducer array is arranged substantially perpendicular to the target area.

18. The implantable ultrasonic transducer device of claim 1, wherein an activation axis of a transducer element of the transducer array is arranged substantially parallel to the target area.

19. The implantable ultrasonic transducer device of claim 1, wherein the transducer array is configured to steer the ultrasonic waves in one or more directions.

20. The implantable ultrasonic transducer device of claim 1, wherein the transducer array is shaped to steer the ultrasonic waves in one or more directions.

21. The implantable ultrasonic transducer device of claim 1, wherein the biometric data is pressure and blood flow rate associated with the target area.

22. The implantable ultrasonic transducer device of claim 1, wherein the antenna is configured to provide the biometric and radiographic data to a 3D or 4D viewing system.

23. The implantable ultrasonic transducer device of claim 1, wherein the transducer device is bio-compatible and magnetic resonance imaging (MRI)-compatible.

24. The implantable ultrasonic transducer device of claim 1, wherein a dura covering of a brain of the patient is sewn directly to the implantable ultrasonic transducer device in order to optimize ultrasound efficacy.

25. The implantable ultrasonic transducer device of claim 8, wherein the diagnostic comprising imaging of at least a portion of the target area.

26. The implantable ultrasonic transducer device of claim 11, wherein the one or more first control signals or the one or more second control signals provided by the controller are configured to activate the transducer array to produce ultrasonic wave of sufficient amplitude, phase, duration to active acoustic dye and record resultant information comprising at least one of: pathophysiologic data, positional data, and metabolic data.

27. The implantable ultrasonic transducer device of claim 1, wherein the implantable ultrasonic transducer device is partially recessed within an intact skull, one's own bone flap following craniotomy and replacement, and/or cranial implant.

28. The implantable ultrasonic transducer device of claim 1, wherein the implantable ultrasonic transducer device contains space for placement of components or devices with synergistic applications comprising at least one of: therapeutic delivery, neuroactivity recording, and hydrocephalus cerebrospinal fluid shunting.

29. The implantable ultrasonic transducer device of claim 1, wherein the transducer array is placed at a tip of an articulating snake.

30. The implantable ultrasonic transducer device of claim 29, wherein the articulating snake is MRI-compatible.

31. The implantable ultrasonic transducer device of claim 29, wherein the articulating snake is manipulated wirelessly in real time or programed to move in a predetermined sequence.

32. The implantable ultrasonic transducer device of claim 29, wherein the articulating snake contains a hollow corridor for passage of objects comprising at least one of: biopsy instruments, high-definition camera, therapeutic delivery systems, and acoustic dye injection.

33. The implantable ultrasonic transducer device of claim 29, wherein the articulating snake length is adjusted to place the articulating snake within deep resection cavities.

34. A computer-implemented method of providing medical services to a patient, the computer-implemented method comprising:
obtaining, by a wireless antenna of an implantable ultrasonic transducer device, one or more first control signals configured to control one or more modes of operation of a transducer array from a client device;
providing, by a controller of the implantable ultrasonic transducer device, one or more second control signals based on the one or more first control signals to a transducer array, wherein the transducer array comprises at least one of diagnostic transducer elements, therapeutic transducer elements, or both;
obtaining, by the controller, one or more response signals based on activation of the transducer array; and
providing, by the wireless antenna, the one or more response signals to the client device for evaluation,
wherein the transducer array, the controller, and the wireless antenna are enclosed within the implantable ultrasonic device and completely contained within a body cavity of a patient and an activation surface of the transducer array is positioned in physical contact with a portion of a treatment area of the patient with no air gap between the activation surface and the treatment area.

35. The computer-implemented method of claim 34, wherein the client device is a smart phone, a laptop computer, a table computer, or a desktop computer.

36. The computer-implemented method of claim 35, wherein the one or more first control signals are obtained by an application operating on the client device.

37. The computer-implemented method of claim 36, wherein the one or more second control signals are determined by the controller to provide an appropriate transducer activation parameter for an operation mode of the one or more modes of operations.

38. A computer-implemented method of providing medical services to a patient via an implantable ultrasonic transducer device, the computer-implemented method comprising:
opening, on a client device, an application to control the implantable ultrasonic transducer device;
selecting, in the application, a patient from among a plurality of patients for treatment;
providing, by the application, one or more control signals configured to control one or more modes of operation of the implantable ultrasonic transducer device; and
obtaining, by the application, one or more response signals based on activation of the implantable ultrasonic transducer device for evaluation,
wherein the implantable ultrasonic device is completely contained within a body cavity of a patient and an activation surface of a transducer array is positioned in physical contact with a portion of a treatment area of the patient with no air gap between the activation surface and the treatment area.
